(19) United States
(12) Patent Application Publication
(10) Pub. No.: US 2021/0244964 A1
(43) Pub. Date: Aug. 12, 2021

(54) SEASONAL AFFECTIVE DISORDER THERAPEUTIC LIGHT SYSTEM

(71) Applicant: Charles Graeber, Nantucket, MA (US)

(72) Inventor: Charles Graeber, Nantucket, MA (US)

(21) Appl. No.: 17/172,504

(22) Filed: Feb. 10, 2021

Related U.S. Application Data
(60) Provisional application No. 62/972,881, filed on Feb. 11, 2020.

(51) Int. Cl. A61N 5/06

(52) U.S. Cl. CPC A61N 5/0618; A61N 2005/0632; A61N 2005/0651

(57) ABSTRACT

A Seasonal Affective Disorder (SAD) treatment system providing seasonally appropriate and aesthetically appealing light therapy in a novel, convenient and medically effective manner. At present, patient compliance has proven to be a significant barrier to the effective treatment of this disorder. Studies have shown that up to 70% of patients report diminished compliance with the therapeutic recommendations using currently available forms of light therapy, in the form of “light boxes”. These patients sited the predominant factor in their failure to comply as shortcomings with the light boxes themselves, finding them “inconvenient” to set up and sit close to for therapeutic periods, or simply too aesthetically “displeasing” to keep in the home or workplace. As a result, as many as 20% of SAD patients who begin light box therapy stop their proscribed SAD lightbox therapy treatment prematurely and entirely. The present invention presents a novel and practical improvement over current therapeutic light-emitting devices by delivering the medically recommended SAD light therapy requirements through more effective, convenient, seasonally appropriate and aesthetically appealing embodiment. By diminishing barriers to patient compliance, this SAD light therapy embodiment facilitates more consistent- and thus more effective-light therapy treatment of SAD, resulting in better therapeutic outcomes for a greater number of those who suffer the debilitating symptoms of this seasonal disorder.

### CROSS REFERENCE TO RELATED APPLICATIONS

The present application claims the benefit of U.S. Provisional Application No. 62/972,881, filed on Feb. 11, 2020, the entire contents of each of the above being incorporated herein by reference.

### TECHNICAL FIELD

The present invention relates generally to a class of electric light-emitting health and personal care devices.

### BACKGROUND

Seasonal Affective Disorder (SAD) is a medically recognized form of seasonal depression. It is believed to be caused by a lack of exposure to sunlight, and as such most typically afflicts sufferers in the fall and winter seasons, when days are shortest and sunlight exposure is minimal. The American Academy of Family Physicians estimates that some four to six percent of the population may suffer from the most severe and debilitating forms of SAD-based “winter depression”, and an additional ten to twenty percent of the population may suffer more mild or occasional seasonal SAD-related symptoms (Am Fam Physician. 1998 Mar. 15; 57(6):1351-1352). According to The Mayo Clinic, signs and symptoms of SAD may include feelings of depression hopelessness and guilt, sluggishness or agitation, sleep issues, low energy, decreased interest in activities once enjoyed, and issues with concentration and mental focus. Symptoms specific to winter-induced SAD may include oversleeping, appetite changes including craving for more carbohydrate-rich foods, weight gain, and feelings of tiredness and low energy. Symptoms may also include frequent thoughts of suicide and death. (https://www.mayoclinic.org/diseases-conditions/seasonal-affective-disorder/symptoms-causes/syc-20364651) The Diagnostic and Statistical Manual of Mental Disorders DSM-IV and DSM-5, recognizes Seasonal Affective Disorder as a specifier for recurrent major depressive disorder, noting that it is a disorder four times more common in women than in men, and that the disorder is more common disorder among populations living in northern latitudes, or those close to the poles, where days are shorter, than in southern latitudes, closer to the equator. For example, SAD is seven times more common among the population living Washington State than in that living in the state of Florida. Other studies have shown that perhaps ten percent of the population of Alaska is seasonally affected (Nolen-Hoeksema, Susan (2014). Abnormal Psychology (6th ed.). New York, N.Y.: McGraw-Hill Education. p. 179. ISBN 978-1-259-06072-4.)

Seasonal affective disorder (SAD), and certain other related conditions, are treatable. The most common and effective therapeutic treatment to alleviate the symptoms of SAD is through timed exposure to bright artificial white light, a therapy more commonly referred to as “lightbox” therapy or “light therapy.” According to the Mayo Clinic, exposure to bright light affects brain chemicals linked to mood and sleep. By mimicking or replacing exposure to natural sunlight, light box therapy may ease a patient's symptoms of SAD and other types of depression, as well as sleep disorders, jet lag. SAD-related fatigue and deficits of mental focus and health problems related stemming from difficulty adjusting to a low sunlight or a nighttime work schedule, such as shift work. In some cases, light box therapy has also shown evidence suggesting possible therapeutic utility in treating some forms of dementia. Light box therapy has also been shown benefit for regulating sleep patterns disturbed by a lack of natural sunlight during winter months.

The effectiveness of this therapy depends on consistent scheduled exposure to light of the proper intensity for a proscribed period of time; a failure to comply with the recommended dose schedule and intensity results in diminished therapeutic results against SAD symptoms. Patient compliance has proven to be a significant barrier to the effective treatment of this disorder. Studies have show that up to 70% of patients report diminished compliance with the therapeutic recommendations, failing to use the light therapy often or long enough. These patients sited the predominant factor in their failure to comply as shortcomings with the light boxes themselves, finding them “inconvenient” to set up, use or access for the proscribed therapeutic periods, or simply too aesthetically “displeasing” to keep in the home or workplace, making regular therapeutic use impractical. As a result, as many as 20% of SAD patients who begin light box therapy stop their proscribed SAD lightbox therapy treatment prematurely and entirely¹. Because light box therapy is associated with the treatment of a recognized psychiatric condition, some patients site privacy concerns, personal embarrassment, and a perceived stigma related to having the lightbox therapeutic device prominently visible in their home or work environment as a barrier to therapeutic compliance. As a result, current light box therapy technologies and iterations fail to provide practical therapeutic utility and effectiveness for a significant percentage of patients seeking treatment for their SAD symptoms. 1 ^{x}Avery, D. H.; Eder, D. N.; Bolte, M. A.; Hellekson, C. J.; Dunner, D. L.; Vitiello, M. V.; Prinz, P. N. (2001). “Dawn simulation and bright light in the treatment of SAD: A controlled study”. Biological Psychiatry. 50(3): 203-216.doi:10.1016/S0006-3223(01)01200-8. PMID 11513820.

During light therapy, a SAD affected individual intentionally exposes themselves to light that enters the eyes indirectly. This light exposure is of a specific therapeutically recommended intensity and duration. Failure to sit or stand in direct exposure to the light box for the proscribed period of time results in loss of potential therapeutic benefit.

The current therapeutic modality (on which the device here described improves), requires that a SAD patient must sit or stand for extended periods near a simple device called a light therapy box (also known as light boxes, bright light therapy boxes and phototherapy boxes) in order to receive the recommended intensity and duration of therapeutic UV exposure. Patients currently have a limited variety of shapes and sizes of light boxes to choose from; some are upright, others small and rectangular, all are fixed solid containments of the light source. This box or device gives off bright light that mimics natural outdoor light.

Therapeutic exposure is specific in duration and intensity. For SAD, the typical recommendation is for a daily 20-30-minute exposure to low or no-UV light with of 10,000 lux intensity. The currently available modality for this therapy is alight box capable of providing the viewer with 10,000 lux when placed a distance of about 16 to 24 inches (41 to 61 centimeters) from the face.

For light therapy to be an effective treatment for SAD and other conditions, physicians recommend it be consistently employed on a regular daily therapeutic schedule. As such, the light therapy device must become a regular feature in the work or home environment for the duration of a person's SAD treatment. As SAD is generally a wintertime condition, the light therapy box or device is usually brought out seasonally, and lived with for those winter months, much in the same way holiday decorations, including seasonal holiday or “Christmas Tree” lights, are brought out for the winter months, then put away and stored during the sunnier seasons.

### SAD Therapy Devices

The most effective form of light therapy device is one that is regularly used. Such a device should be 1. easily, effectively and pleasingly integrated into the home or work environment, especially a home or work environment as laid out in the winter season and 2. convenient to be most easily consistently employed by a patient as part of a regular recommended light therapy routine, as medically suggested for effective therapeutic effect. The ideal light therapy device would serve as a functional and non-obtrusive aspect of the regular home or office environment, allowing the user to employ the device regularly, without stigma and at their convenience.

Barriers to Patient Compliance and Therapeutic Benefit

Any practical, emotional, social or aesthetic factors which impede, hinder or prevent a patient's ability or willingness to integrate a light therapy device (i.e. a therapeutic device for the treatment of a psychiatric condition) into the home or workplace environment diminish that patient's practical access, utility and use of the therapeutic device, and thus diminish the consistency, duration or employment of the therapy and any positive therapeutic effects such a device or therapy might convey or confer.

Currently available light therapy devices embody many of these negative factors. Most light boxes are private medical devices which are considered by many users to be unattractive, unwieldy, inconvenient and, for some, embarrassing Yet during the season of their therapeutic use these therapeutic devices must occupy a regular place in our homes and workplaces among objects and décor carefully chosen to provide joy rather than displeasure or embarrassment. Their employment also may not be practical, especially in a work environment, where the psychiatric therapeutic associations of the device may betray medical privacy and may publicly stigmatize the user, jeopardizing his or her reputation as a reliable and mentally fit employee. During the season of their therapeutic employment, these devices also take up additional counter or floor space in the home or office, adding to their inconvenience. As a result of these negative factors, conventional lightbox therapeutic devices are typically stowed or hidden for the majority of the day, when not in use, and only brought out and set up situationally for the therapeutic period. Symptoms of SAD frequently involve feelings of low energy and difficulties in concentrating on daily tasks, magnifying the difficulty in regularly employing the therapeutic device. The recipient of the light box therapy must also remain in close proximity to the light box in order to receive therapeutic levels of light. As the light box is commonly placed at head-height on a desk and has a range of therapeutic effectiveness of approximately 24 inches from the viewer's face, any deviation from therapeutic position, including standing, moving around the room, or even leaning back in one's chair, has the effect of removing the viewer from effective therapeutic proximity. This limitation, compounded by the daily inconvenience and additional time and planning required to retrieve, set up, employ, tear down and put away the current light therapy iteration, often results in shortened, missed or skipped therapeutic sessions, incomplete therapeutic intensity due to inconsistent placement of the device during daily setup, and inconsistent or irregular proximity of the patient to the lightbox. In sum, the current lightbox therapy technology makes it difficult for many patients to successfully receive the consistent administration of light therapy medically recommended for the effective treatment of SAD.

The net result of the current limitations of lightbox therapy often result in inconsistent timing, duration and intensity of the therapy, and may act as a disincentive toward consistent compliance with the therapeutic regimen. As a result, light box therapy often proves inefficacious for many who would otherwise benefit from bright light therapy for the treatment of Seasonal Affective Disorder.

### SUMMARY OF THE INVENTION

The present invention solves the above deficiencies by providing unique and practical improvements over current therapeutic lightboxes and light-emitting devices used in the treatment of SAD. It does so by delivering the medically recommended therapeutic light intensity through a novel, more effective, convenient, seasonally-appropriate and aesthetically appealing embodiment—one which integrates seamlessly into the home or office décor, and which may be readily employed on a regular basis without embarrassment or social stigma. In fact, this embodiment mirrors one that already serves a decorative function in many homes during the winter months, adding desired SAD therapeutic value to an embodiment currently commonly employed in many homes for purely aesthetic purposes. By diminishing barriers to patient compliance, this SAD light therapy embodiment facilitates more consistent, and thus more effective treatment of SAD, resulting in better therapeutic outcomes.

An objective of the present invention is to provide those suffering from SAD with a novel, convenient and effective device for receiving light therapy, providing improved relief from the debilitating symptoms of the condition while removing the barriers of stigma and inconvenience which prevent the majority of SAD sufferers from successfully receiving treatment.

In one embodiment, the SAD therapeutic light system comprises a driver to convert AC voltage to DC, a standard three-prong AC plug, and multiple light emitting diodes (LEDs) sufficient to generate therapeutic intensity of low or no-UV light, wherein the multiple LEDs are arranged in an array pattern. The LEDs may have a lumen rating of 45, 705 and 1005 lumens. LEDs with other lumen ratings are also contemplated. Depending on the lumen rating of the LEDS, the array may comprise 83, 64 or 45 LEDs. The array may be in the shape of a triangle, rectangle or any other geometric shape. This LED array is sufficient to produce 15,000 lux of illumination for the viewer at maximum power and optimized orientation, such that even under realistic sub-optimal orientation to or distance from the viewer, a therapeutic level of light intensity may be realized.

In another embodiment, the SAD therapeutic light system comprises a driver to convert AC voltage to DC and a standard three-prong AC plug and multiple light emitting diodes (LEDs) sufficient to generate therapeutic intensity of low or no-UV light, wherein the multiple LEDs may be arranged in a string configuration. This LED array is sufficient to produce 15,000 lux of illumination for the viewer at maximum power and optimized orientation, such that even under realistic sub-optimal orientation to or distance from the viewer, a therapeutic level of light intensity may be realized.

### DESCRIPTION OF THE DRAWINGS

The foregoing summary, as well as the following detailed description of the invention, will be better understood when read in conjunction with the appended drawings. For the purpose of illustrating the invention, the drawings show those embodiments that are presently preferred. It should be understood, however, that the invention can be embodied in different forms and thus should not be construed as being limited to the embodiments set forth herein.

FIG. 1 depicts various electrical arrangements of the present invention;

FIG. 2 is an embodiment of a SAD Therapeutic light system of the present invention;

FIG. 3 is a second embodiment of a SAD Therapeutic light string of the present invention with Brightness/Mode Control;

FIG. 4 is an embodiment of a SAD Therapeutic light system array of the present invention;

FIG. 5 is another embodiment of a SAD Therapeutic light system array of the present invention; and

FIGS. 6A through 6C are views of the embodiments depicted in FIGS. 2 and 5 in a home environment.

### DETAILED DESCRIPTION

The present subject matter will now be described more fully hereinafter with reference to the accompanying figures, in which representative embodiment(s) are shown. The present subject matter can, however, be embodied in different forms and should not be construed as limited to the embodiment(s) set forth herein. Rather, these embodiments are provided to describe and enable one of skill in the art. Unless otherwise defined, all technical and scientific terms used herein have the same meaning as commonly understood by one of ordinary skill in the art to which the subject matter pertains. All publications, patent applications, patents, and other references mentioned herein are incorporated by reference in their entirety.

The invention described herein removes barriers to SAD patient compliance by providing therapeutic levels of light therapy via an aesthetically pleasing and more convenient, effective and practical iteration than the light therapy devices currently available.

The invention delivers low or no-UV light that is variable, via controller, from purely ambient (low) to therapeutic levels (i.e. at or above 10,000 lux), via an arrangement of LED's (arranged in string and net/array lighting configurations and resembling seasonal, holiday or “Christmas Tree” style lighting) that integrates conveniently and practically as a regular feature of the home or work environment, facilitating greater therapeutic control over light dosage, more regular patient access to the palliative light therapy, and increased compliance with, and results from, the medical recommendations for treatment of Seasonal Affective Disorder and other conditions for which such light therapy is considered efficacious.

The embodiments of this invention deliver therapeutic-levels of low or no-UV light to the viewer/patient. The configuration of the light emitting diodes (LEDs, also referred to as diodes in this specification) are a product of variables including the type, power and number of diodes employed, the proximity of those diodes to one another along the string or in the array, the overall length of that string/wire, (size of array) and the distance of the viewer/patient from the light source.

The diodes used in the embodiments have optimal viewing angles of 115-120 degrees. To account for the possibility of less-than-optimal efficiency of LED orientation to the viewer and ensure that therapeutic levels are reached, the embodiments described in the following tables produce 15,000 lux, substantively more than the 10,000 lux medical experts describe as being the minimum required for efficacious light therapy for the treatment of SAD. Controllers described elsewhere in this specification allow the viewer to modify the exact lumens or brightness of the embodiments received to best match their therapeutic needs and to compensate for greater distance or proximity as regards the embodiments, or any physical barriers between the invention and the viewer/patient which may otherwise prevent them from receiving therapeutic levels of light. Such controls may also be employed in order to increase or lower the intensity of lumens between periods of therapeutic dosage and other, non-therapeutic periods of utility of the light string or array, such as purely decorative or functional uses.

The following tables provide the configurations of the embodiments based on the LED the number of LEDs, length of wire and spacing of those diodes along that wire needed to create embodiments of the invention that generate therapeutic lumen intensity sufficient to reach a viewer at distances of 6 feet and 10 feet. For clarity a preferred length of string of 15 feet is used in these examples. The string length can in practice be greater or less than 15 feet. If no net changes in total lumens produced are desired, changes to the length of the string will impact the spacing of the diodes along that length, i.e., on a longer string, the same diodes would be spaced further apart in order to be evenly distributed on the string and produce the same amount of light. Aesthetic as well as therapeutic considerations factor into the weighing variables of diode type and intensity, the distance between diodes and the total length of string.

The preferred LEDs are 545 lumen diodes, producing 15,000 lux at 6 feet. As noted previously, the embodiments create greater lux than required for therapeutic levels in order to compensate for any inefficiency in the orientation of the LEDs to the viewer, and as in embodiments 2 through 4 the intensity may be raised or lowered by the user situationally, in order to ensure that they are receiving therapeutic levels.

Table 1 below demonstrates the relationship between the number of diodes required, the overall length of wire those diodes are strung along, distance between each diode and net power requirements utilizing 545 lumen diodes and capable of delivering 15,000 lux at various distances.

For example, in order to effectively provide therapeutic levels (>10,000 lux) at a distance of 6 feet, this table shows that the diodes of 545 lumens each are spaced 3.3 inches apart along a wire 15 feet in length. A total of 56 diodes are required, drawing 252 Watts. For effectiveness at 10 feet, those 545 lumen diodes need to be spaced 2.2 inches apart along the same length of wire, requiring a total of 83 diodes and 373.5 Watts.

TABLE 1

| Power Requirements per | | | 500 mA | 9 V | 4.5 W |
|---|---|---|---|---|---|
| diode | | | | | |
| Thermal Resistance | | | 1.9 C/W | | |
| Target Lux: | 15,000 | | | | |
| Luminous Flux Per Diode | 545 | lumens | | | |
| Diodes Needed to be | 56 | | | | |
| Therapeutically Effective | | | | | |
| at 6 feet | | | | | |
| Diodes Needed to be | 83 | | | | |
| Therapeutically Effective | | | | | |
| at 10 feet | | | | | |
| Wattage, 6 feet | 252 | Watts | | | |
| Wattage, 10 feet | 373.5 | Watts | | | |
| Heat Per Diode | 8.55 | Celsius | | | |
| For 6-loot effectiveness: | | | | | |
| Stung length (in feet) | 15 | | | | |
| Diode Spacing (in inches) | 3.3 | | | | |
| For 10-foot effectiveness: | | | | | |
| String Length (in feet) | 15 | | | | |
| Diode Spacing (in inches) | 2.2 | | | | |

More powerful diodes may also be used to produce a therapeutic intensity of light at various distances, as the tables below demonstrate. The variables of distance between diodes, overall length of string and power requirements are altered by using these higher lumen diodes.

Table 2 demonstrates the relationship between the number of diodes required, the overall length of wire those diodes are strung along, distance between each diode and net power requirements for delivering 15,000 lux at various distances utilizing 705 lumen diodes.

In order to effectively provide therapeutic levels (>10,000 lux) at a distance of 6 feet, the diodes of 705 lumens each need to be spaced 4.2 inches apart along a wire 15 feet in length. This configuration requires 43 diodes in total and draws 361.2 Watts. For effectiveness at 10 feet, those diodes would be spaced 2.2 inches apart along the same length of wire, requiring a total of 64 diodes and 573.6 Watts.

TABLE 2

| Power Requirements per diode | | 700 mA | 12 V | 8.4 W |
|---|---|---|---|---|
| Thermal Resistance | | 1.2 C/W | | |
| Target Lux: | 15,000 | | | |
| Luminous Flux Per Diode | 705 | lumens | | |
| Diodes Needed to be Therapeutically | 43 | | | |
| Effective at 6 feet | | | | |
| Diodes to be Therapeutically | 64 | | | |
| Effective at 10 feet | | | | |
| Wattage, 6 feet | 361.2 | Watts | | |
| Wattage, 10 feet | 537.6 | Watts | | |
| Heat Per Diode | 10.08 | Celsius | | |
| For 6 foot effectiveness: | | | | |
| String Length (in feet) | 15 | | | |
| Diode Spacing (in inches) | 4.2 | | | |
| For 10-foot effectiveness: | | | | |
| String Length (in feet) | 15 | | | |
| Diode Spacing (in inches) | 2.9 | | | |

Table 3 demonstrates the relationship between the number of diodes required, the overall length of wire those diodes are strung along, distance between each diode and net power requirements for an embodiment capable of delivering 15.000 lux at various distances utilizing 1005 lumen diodes.

To effectively provide therapeutic levels (>10,000 lux) at a distance of 6 feet, the configuration uses diodes of 1005 lumens each and spaced 6 inches apart. This configuration requires 30 diodes in total and draws 216 Watts. For effectiveness at 10 feet, those diodes would be spaced 4 inches apart along the same length of wire, requiring a total of 45 diodes and 324 Watts.

TABLE 3

| Power Requtrements per diode | | 800 mA | 9 V | 7.2 W |
|---|---|---|---|---|
| Thermal Resistance | | 3.2 C/W | | |
| Target Lux: | 15,000 | | | |
| Luminous Flux Per Diode | 1005 | lumens | | |
| Diodes Needed to be Therapeutically | 30 | | | |
| Effective at 6 feet | | | | |
| Diodes Needed to be Therapeutically | 45 | | | |
| Effective at 10 feet | | | | |
| Wattage, 6 feet | 216 | Wafts | | |
| Wattage, 10 feet | 324 | Watts | | |
| Heat Per Diode: | 23.04 | Celsius | | |
| For 6-foot effectiveness: | 15K | | | |
| String Length (in feet) | 15 | | | |
| Diode Spacing (in inches) | 6 | | | |
| For 10-foot effectiveness: | | | | |
| String Length (in feet) | 15 | | | |
| Diode Spacing (in inches) | 4 | | | |

For embodiments utilizing a net-like array of wires along which a rectangular grid of LEDs is configured, Table 4 provides examples of the number of diodes, distance between diodes and total length of horizontal wire required to enable a viewer at distance at 10 feet to receive lumens considered medically sufficient for the therapeutic treatment of Seasonal Effective Disorder.

Table 4 provides the density, number and spacing of 545, 745 and 1005 lumen diodes respectively, wired into an array or net-like grid, with variable weave of X and Y axis wiring possible (the spacing between horizontal and vertical wires to create the grid). One configuration creates a basic grid consisting of 5 wires in total, overlayed perpendicularly to create a net-like arrangement. For the purposes of this example, calculations for an array capable of generating 15,000 lux at 10 feet are shown. Distances within the table are shown in inches:

TABLE 4

| | | | | LEDs | | |
|---|---|---|---|---|---|---|
| | # of | # of | Wire | per | LED | |
| LED | Wires | LEDs | Length | Wire | Spacing | Power |
| 545 Lumen | 5 | 83 | 60 | 17 | 3.5 | 373.5 W |
| 705 Lumen | 5 | 64 | 60 | 11 | 5.4 | 537.6 W |
| 1005 Lumen | 5 | 45 | 60 | 9 | 6.6 | 324 W |

LED spacing is both horizontal and vertical

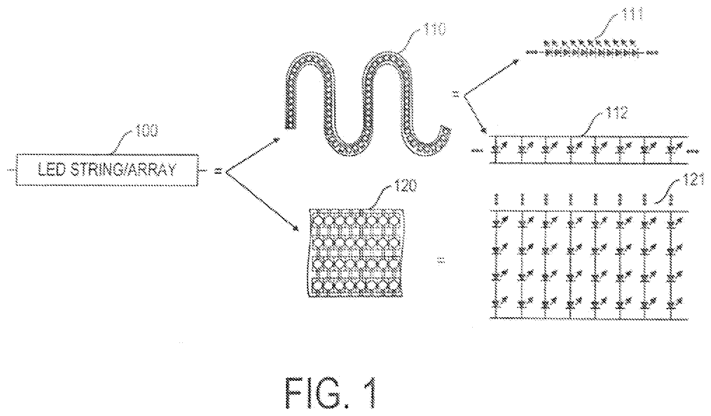

FIG. 1 depicts various electrical implementations of the present invention, such that the present invention 100 may comprise an LED string configuration 110 or array (net) configuration 120. A number of design considerations are necessary for one or more implementations of the invention.

Rows or strings of LEDs can be configured in any of several possible “electrical” arrangements—series, parallel, and series/parallel topology to use. The most common LED topologies are serial Ill and parallel arrangements 112 (FIG. 1). For 111, the Anode is to the left, and the cathode is to the right of each diode. For 112, the anode is on top, and the cathode is on the bottom. These serial/parallel arrangements may be combined to yield two other orientations, series/parallel (not shown) and parallel/series 121 (FIG. 1). The LEDs in the array have their anodes connected to switched power strings and their cathodes connected to individually switched grounds.

The two arrangements—series string, series/parallel, and all be operated with “analog” control. Such analog control systems do not need digital processors to control their illumination.

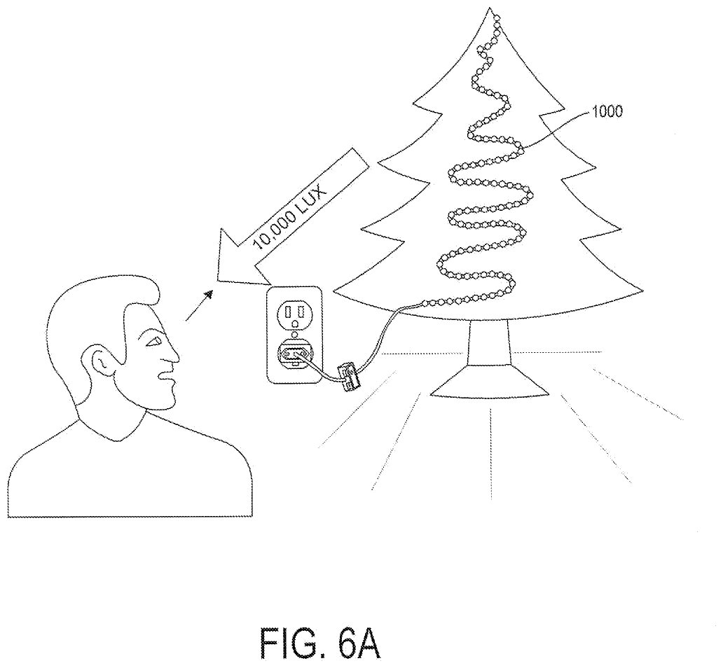

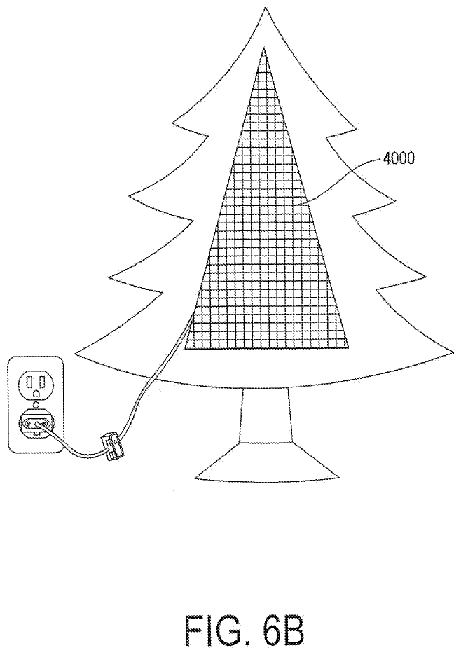

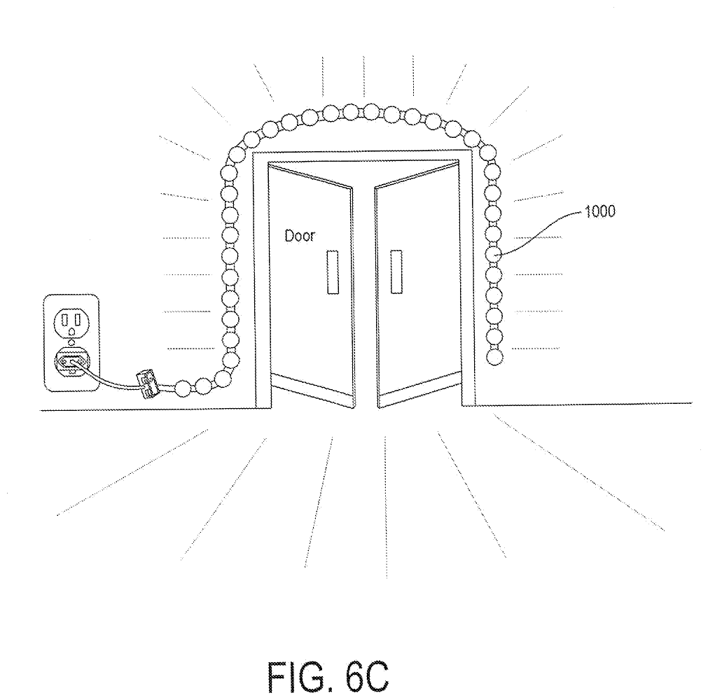

Beyond any particular electrical arrangement of LED strings, the physical arrangement is an increasingly important characteristic, as discussed above. The LED string be flexible to allow the string(s) to wrap around or be draped over an area of the surface of a Christmas tree (See FIG. 6A) or other object in a home or office or decorate the interior of a room (See FIG. 6C). Similarly, an LED array must be similarly flexible to allow for it to be draped on or around a Christmas tree or other objects. (See FIG. 6B) As more LEDs are used, string flexibility and “chaining” of strings becomes more important.

Of the two basic kinds of LED strips, analog and digital, the analog-type strip has the LEDs connected in a serial/parallel configuration.

To regulate the power to the LED string or strings, the present invention includes an LED driver. Many different LED drivers are commercially available. The output of the LED driver matches the electrical characteristics of the LEDs. Depending on the configuration of the present invention, LED drivers may provide dimming of the LED string and may have more than one channel for separate control of different LEDs. The power level of the LED is maintained constant by the LED driver. A controller, shown in embodiments 2 through 4, is an interface between the power supply and LEDs. The controller allows a user to manipulate the LED properties. For instance, it could turn on/off individual strings of LEDs, flash LEDs, set LED string brightness (dimming), for color LEDs it could tune the output of the LEDs to designated wavelengths, and it could act as a timer to turn lights on or off at specific times of the day. An additional “mode button” may be integrated, as is common in such holiday-type string lighting, in order to allow the user to change the lighting display to flash off and on, fade in and out periodically, twinkle, etc. Such modes are a common feature in the currently commercially available holiday light strings.

It is envisioned that the controller can be “manual” in that the user by means of knobs or buttons or switches associated with the controller selects LED characteristics. Other embodiments of the invention may include an “automatic” or digital controller. In these embodiments multiple LED operating parameters could be programmed into a microcontroller or similar device.

Drivers with dimming capability can dim the LED light output over the full range from 100% to 0%. Dimming drivers can dim LEDs by reduction in the forward current, pulse width modulation (PWM) via digital control, or more sophisticated methods. Most dimming drivers operate using the PWM method. LED dimming is accomplished either by PWM dimming or constant current dimming. Constant current dimming has the advantage of creating illumination intensities which are non-flickering but requires more circuitry per channel than PWM dimming. Constant current dimming can result in poor color mixing with RGB LEDs at high currents and does not perform well under 10% brightness in any application. A new class of drivers can use PWM under 10% and constant current dimming over 10% brightness, making this the preferred embodiment.

The LED's physical arrangement determines the electrical arrangement from which a Driver/Controller is determined. It is envisioned that an external LED driver is utilized. However. LED strings having built-in drivers are also contemplated. High powered LEDs have a ‘luminous flux’ brightness rating in lumens. To convert this to lux, one must measure the space that the LEDs will illuminate. A high-power LED whose luminous flux measures 500 lumens will deliver 250 lux to illuminate a 2 square meter area. The number of LEDs required to achieve 10K lux will vary based on the area that needs to be illuminated, and the particular LED chosen for the application (see discussion above).

The LED string/array may be powered with an AC voltage source such as a common wall outlet of 60 Hz and 115 VAC or 230 VACS

### Embodiment 1

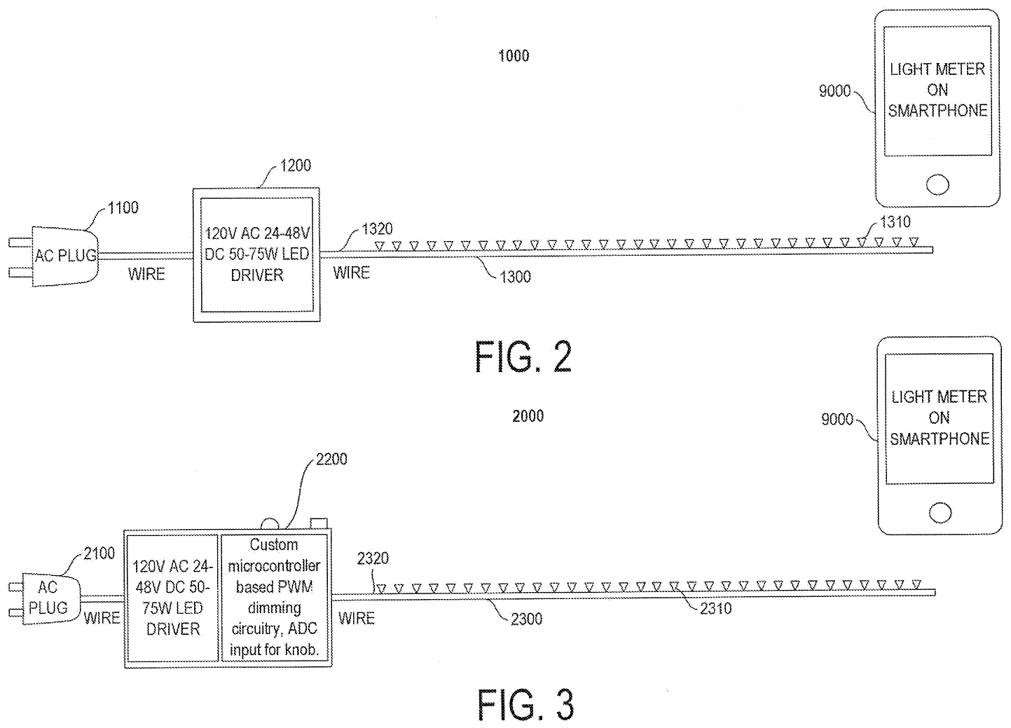

FIG. 2 depicts a first embodiment of the SAD lights 100 of the present invention. Depending on the brightness or lumen-generating capacity of the LED, an LED string may consist of between thirty (30) to eighty-three (83) diodes (LEDs), depending on the target lux as described in Tables 1, 2, and 3. The LED string may be draped on or around a Christmas tree (see FIG. 6A) or other similar bush or plant in a typical decorative holiday manner, or similarly festively lining the wall/ceiling, furnishings, mirrors, or other features of home décor indoors or out. The total length of each LED or Light string ranges between approximately 5 feet to 30 feet with a preferred length of 15 feet. Other lengths of LED strings are contemplated, with corresponding increases of diodes required. The first embodiment 1000 (FIG. 2) includes an AC plug 1100, and a commercially available LED driver 1200 capable of driving a LED strip 1300 having the capability of handling the power disclosed in tables 1, 2 and 3. The AC plug 1100 is compatible with standard wall sockets. The LED Driver may convert the AC voltage to a DC voltage. The LEDs 1310 may draw between 500 and 800 mA. This is dependent on the type of LED employed. The Light String would be utilized therapeutically by arranging these string lights on an indoor or outdoor arrangement, such as a tree or indoor plant or in a string or design framing a door or on a wall. The terms Light String and LED String are used interchangeably, and that the length is the distance from the first LED to the last LED and does not include driver/controller, AC plug or the wire connecting the Light String to the driver/controller and AC plug. A person afflicted with SAD can use a light meter application on his/her smartphone 9000 or other similar device or devices performing a similar function (not part of the invention), in order to help determine lux at any position relative to the device, and thus the optimal distance, orientation and exposure time as regards the LED string for obtaining the desired benefit of the SAD therapy. In embodiments of the SAD driver which allow for adjustments in lumen output, a smartphone app or utility performing a similar function may also be employed to make such adjustments, increasing or decreasing the intensity of light and timing the duration of that exposure to ensure that therapeutic lumen levels optimized to the viewer's distance and position.

The AC plug 1100 is preferably a 3-prong plug with aground wire, such as a NEMA 1-15 or a 5-15 plug. The AC wire 1320 is preferably a jacketed 18 AWG stranded wire. The LED driver is a 120 VAC 24-48-volt DC output constant current LED driver that is commercially available.

### Embodiment 2

FIG. 3 depicts an electrical diagram of a second embodiment 2000 of the present invention. The second embodiment 2000 may include additional control features of the SAD lights of the first embodiment 1000 of the present invention. The AC plug 2100, LED strip 2300, LEDS 2310 and AC wire 2320 are similar or the same as those found in the first embodiment. It may also include colored lights, or LEDs capable of changing frequency and color, for aesthetic and festive/decorative purposes, as well as other possible therapeutic (e.g., red light therapy purposes). As in FIG. 1, the embodiment in FIG. 2 may consist of a string of high-power LEDs 2300. The total length of each LED string or light string ranges between approximately 5 feet to 30 feet with a preferred length of 15 feet. Light strings may be made available in greater lengths with greater number of LEDs in total, effectively extending the total length and/or increasing the potential maximum total light intensity (lux) delivered. The second embodiment (FIG. 3) includes an AC plug 2100, and an LED driver 2200 capable of driving 50 to 75 watts LEDs at 24 to 48 volts. The LED Driver 2200 may power a string 2300 of thirty (30) to eighty-three (83) high power LEDs 2310. The LEDs 2310 draw between 500 and 800 mA each. This is dependent on the type of LED employed (see discussion above with respect to Tables 1, 2 and 3). The total light emitted is at least 15,000 lux, ensuring the viewer will receive an intensity of low-UV light medically accepted as one of sufficient therapeutic intensity. After arranging these string lights on an indoor or outdoor arrangement, such as a tree or indoor plant, the person afflicted with SAD can use a light meter application on his/her smartphone 9000 or other similar device or devices performing a similar function (not part of the invention), in order to help determine the optimal distance, orientation and exposure time as regards the LED string for obtaining the desired benefit of the SAD therapy. As smart phones commonly have sophisticated digital cameras built into their designs, these cameras are already engineered to be precise light meters, and commonly available apps, such as Lux Light Meter Pro, provide both an accurate handheld measure of lumens from any given location, as well as a running tally of total lumens received over time. This modality is ideal for the user of this SAD light array device to measure their lumen exposure and ensure that they receive the therapeutic level of full spectrum light over the course of their movements around the room in which the device is employed. To further facilitate the receipt of the therapeutically suggested lumens from this device over time, the intensity of the lumens may be adjusted. In this embodiment, duration and intensity may be altered by increasing or decreasing the light intensity via controller, or “dimmer” which is incorporated into the LED Driver 2200. The dimmer may be one of various forms, sliding, dialing, or digital; it may be purely physically embodied on the cord, either near or at the power source and plug or incorporated into the string of LEDs, or it may be used via a paired remote control, or controlled as a “smart device” via WIFI or other similar electronic communication paradigm. The result allows for the device to serve as an aesthetically pleasing and practical aspect of daily home décor and interior lighting that may be also be conveniently transitioned or “dialed up” into a therapeutic function for regular SAD light therapy.

### Embodiment 3

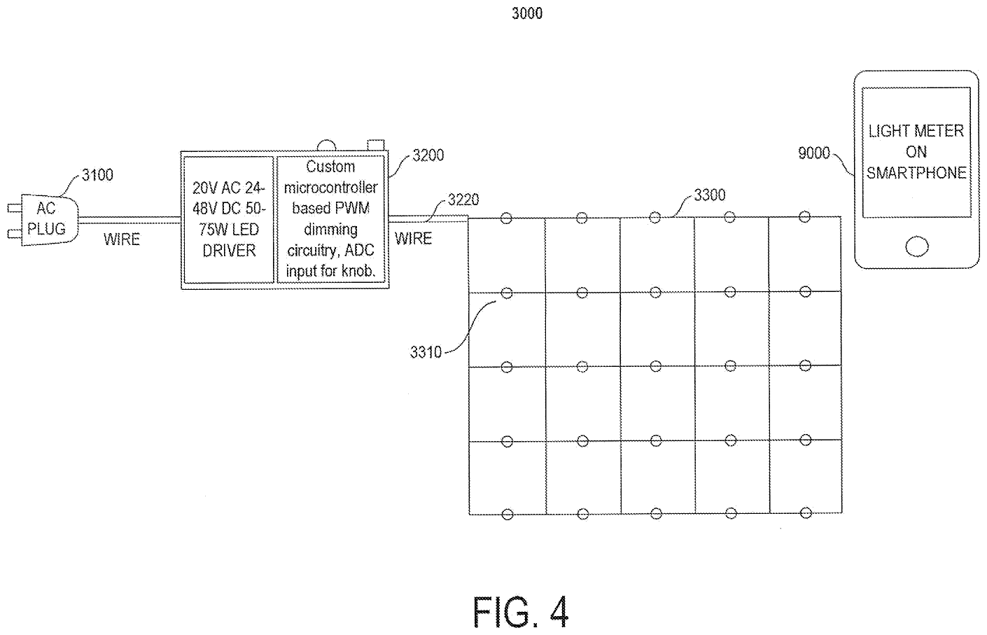

FIG. 4 depicts a third embodiment 3000 of the present invention. This embodiment may have the LEDs configured in an array formation instead of a linear string of LEDs. This array is a fixed or net-like configuration of the conducting strings of LEDs in relation to one another, resulting in a net-like pattern or array within a pre-determined shape of fixed dimensions, with a set density of LEDs within that shape. The shape of such an array may be conical or pyramidal, or the net-like array may be square or squared, or rectangular. The array is a grid or otherwise repeated physical matrix of equally spaced LEDs, either on wires running horizontally, vertically (not shown), or both (not shown), and can be controlled via a dimmer, increasing or decreasing the brightness. In terms of the number of diodes and their spacing from one another, one may think of the array as a perfectly organized version of the string embodiment, with some segments of that string running horizontally, others vertically, creating a familiar grid or net-like array, along which diodes are evenly distributed. The size of each square in this grid, and the overall size of the array itself, may be determined by several factors, including aesthetic concerns, as well as the density of diodes required within the array in order to produce therapeutic levels of light. That requirement, and the specifics of the size of the grid and overall array, are all factors that impact the spacing of the diodes along that array (See Table 4 above). Other non-therapeutic features may be incorporated into the array including various holiday light programs which result in twinkling or fading or vacillating light forms, all of which may be controlled via the base module, a remote control, of via an app (not part of invention), through WIFI or a similar means. SAD therapeutic LEDs and non-therapeutic, purely decorative lights (“holiday” or “Christmas” lights, also sometimes referred to as “fairy lights”) may be co-mingled or alternated on the strings and may be controlled separately or in concert. The benefit of the net array is the regularity of the spacing of the LEDs, which allows the SAD LED to be thrown or draped over a tree or bush (such as an evergreen, or a Christmas tree) providing blanket coverage, or draped, placed or hung on a home feature such as a wall or curtain, establishing an LED grid with a consistent intensity of therapeutic light emanating from the chosen surface. While the net-like arrangement of light strings is the preferred embodiment, other embodiments are contemplated, including zigzag or other light arrangements within a triangular or pyramidal external frame, or with the diodes linearly on top of or within the array of flexible plastic, netting or substrate. In this case, the flexible frame would be non-conductive and separate from the wired strings within it, serving only to anchor the extremities of the array within its form, and provide structure for the array to be hung or draped over an object such as a bush or Christmas tree, or hung independently on a flat surface such as a wall without losing its form or putting additional stress upon the circuitry.

The third embodiment 3000 may include additional control features of the SAD lights of the first embodiment 1000 of the present invention. The AC plug 3100, LED array 3300, LEDS 3310 and AC wire 3320 are similar or the same as those found in the first and second embodiments. It may also include colored lights, or LEDs capable of changing frequency and color, for aesthetic and festive/decorative purposes, as well as other possible therapeutic (e.g., red light therapy purposes). Each of the diodes themselves may also be covered or encased within an opaque or translucent diffuser bulb or shell to increase the viewing angle of each LED, alter or add color or effect, including prismatic or chromatic effects, or to filter, distribute, dissipate or diminish the luminous intensity of the individual diode. This feature may be included in the other embodiments as well.

### Embodiment 4

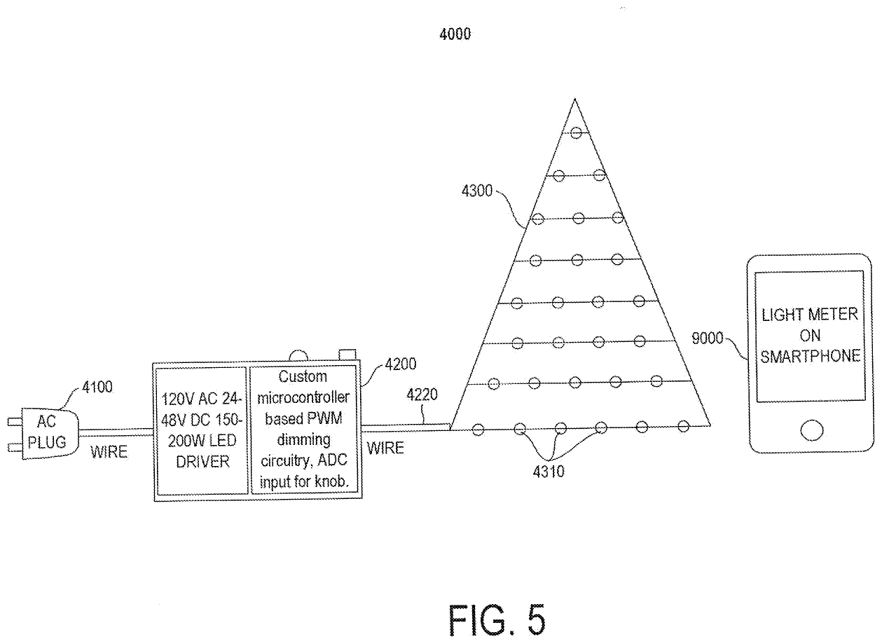

FIG. 5 depicts a fourth embodiment 4000 of the present invention. This embodiment may have the LEDs configured in a triangular array formation instead of a square or rectangular array of LEDs. This array is a fixed or net-like configuration of the conducting strings of LEDs in relation to one another, resulting in a net-like pattern or array within a pre-determined shape of fixed dimensions, with a set density of LEDs within that shape. The array is a grid (vertical wiring lines are not shown for reasons of clarity but are depicted in FIG. 6B) of equally spaced LEDs, and can be controlled via a dimmer, increasing or decreasing the brightness.

Other non-therapeutic features may be incorporated, including various holiday light programs which result in twinkling or fading or vacillating light forms, all of which may be controlled via the base module, a remote control, of via an app (not part of invention), through WIFI or other control. SAD therapeutic LEDs and non-therapeutic, purely decorative lights (“holiday” or “Christmas” lights) may be co-mingled or alternated on the strings and may be controlled separately or in concert. The benefit of the net array is the regularity of the spacing of the LEDs, which allows the SAD LED to be thrown or draped over a tree or bush (such as an evergreen, or a Christmas tree) providing blanket coverage, or draped, placed or hung on a home feature such as a wall or curtain, establishing an LED grid with a consistent intensity of therapeutic light emanating from the chosen surface. A triangular shaped array allows the array to conform to the shape of a Christmas tree, ensuring the LED's illumination is oriented within the viewer's optimal viewing angle of between 115-120 degrees. This arrangement is designed to ensure that the array is situated exclusively on the portion of the tree most proximate to and oriented toward the viewer, ensuring that the maximum number of LEDs in the array are directed toward and illuminating the viewer and that all of the LEDs are contributing to the total levels of therapeutic illumination.

The fourth embodiment 4000 may include additional control features of the SAD lights of the first embodiment 1000 of the present invention. The AC plug 4100, LED array 4300, LEDS 4310 and AC wire 4320 are similar or the same as those found in the first and second embodiments. It may also include colored lights, or LEDs capable of changing frequency and color, for aesthetic and festive/decorative purposes, as well as other possible therapeutic (e.g., red light therapy) purposes. Each of the diodes themselves may also be covered or encased within an opaque or translucent diffuser bulb or shell to increase the viewing angle of each LED, alter or add color or effect, including prismatic or chromatic effects, or to filter, distribute, dissipate or diminish the luminous intensity of the individual diode.

The LED Driver 4200 may power an array 4300 consisting of forty-five (45) to eighty-three (83) diodes 2310. The LEDs 2310 draw between 545 and 1000 mA each. This is dependent on the type of LED employed. LEDs may be spaced between 1 to 3 inches from each other on the array. The total intensity of lumens generated is at least 15,000 lux, such that a viewer will receive the 10,000 lux or greater intensity of low-UV or no-UV light medically recommended as providing therapeutic benefit for the treatment of Seasonal Affective Disorder. After arranging the array of LEDs on an indoor or outdoor arrangement, such as a tree or indoor plant, the person afflicted with SAD can use a light meter application on his/her smartphone 9000 or other similar device or devices performing a similar function (not part of the invention), in order to help determine the optimal distance, orientation and exposure time as regards the LED string for obtaining the desired benefit of the SAD therapy. As smart phones commonly have sophisticated digital cameras built into their designs, these cameras are already engineered to be precise light meters, and commonly available apps provide both an accurate handheld measure of lumens from any given location, as well as a running tally of total lumens received over time. This modality is ideal for the user of this SAD light array device to measure their lumen exposure and ensure that they receive the therapeutic level of full spectrum light over the course of their movements around the room in which the device is employed. To further facilitate the receipt of the therapeutically suggested lumens from this device over time, the Intensity of the lumens may be adjusted. In this embodiment, duration and intensity may be altered by increasing or decreasing the light intensity via controller, or “dimmer” which is incorporated into the LED Driver 3200. The dimmer may be one of various forms, sliding, dialing, or digital, it may be purely physically embodied on the cord, either near or at the power source and plug or incorporated into the string of LEDs, or it may be used via a paired remote control, or controlled as a “smart device” via WIFI. This control may take the form of an application on a smart phone which allows the user to monitor their light exposure time and modify the light device in intensity and program. When the SAD light therapy devices is integrated along with other holiday lights, as part of a Christmas tree, the controller could be used to dial down the intensity of the SAD lights so as to match the other non-therapeutic lights when the device is not in use for SAD therapy, or it may be programmed to act as a sort of artificial sunrise and sunset, slowly increasing in illumination as an alarm clock during otherwise dark morning hours and helping to regulate circadian rhythm by simulating the longer hours of light exposure typified by longer summer days. The result allows for the device to serve as an aesthetically pleasing and practical aspect of daily home décor and interior lighting that may be also be conveniently transitioned or “dialed up” into a therapeutic function for regular SAD light therapy.

Although the present invention has been described in considerable detail with reference to certain embodiments thereof, other embodiments are possible. Therefore, the spirit and scope of the appended claims should not be limited to the description of the embodiments contained therein. It should be understood that various changes, substitutions, additions and alterations can be made by one skilled in the art without departing from the spirit and scope of the invention as defined by the appended claims. Moreover, the scope of the present application is not intended to be limited to the particular embodiment(s) of the device, process, machine, manufacture and composition of matter, means, methods and or steps described in the specification. As one of ordinary skill in the art will readily appreciate from the disclosure herein, processes, machines, manufacture, composition of matter, means, methods or steps, presently existing or later to be developed that perform substantially the same function or achieve substantially the same result as the corresponding embodiments described herein may be utilized according to the present invention.

1. A therapeutic light system comprising:
a driver to convert AC voltage to DC;
a standard three-prong AC plug; and
multiple light emitting diodes (LEDs) which produce 15,000 lux of illumination,
wherein the multiple LEDs are arranged in an array pattern.

2. The therapeutic light system of claim 1, wherein the LEDs have a lumen rating of 545 lumens.

3. The therapeutic light system of claim 2, wherein the array comprises 83 LEDs.

4. The therapeutic light system of claim 1, wherein the array is in the shape of a triangle.

5. The therapeutic light system of claim 1, wherein the LEDs have a lumen rating of 705 lumens.

6. The therapeutic light system of claim 5, wherein the array comprises 64 LEDs.

7. The therapeutic light system of claim 1, wherein the LEDs have a lumen rating of 1005 lumens.

8. The therapeutic light system of claim 7, wherein array comprises 45 LEDs.

9. A therapeutic light system comprising:
a driver to convert AC voltage to DC;
a standard three-prong AC plug; and
multiple light emitting diodes (LEDs) which produce 15,000 lux of illumination,
wherein the multiple LEDs are arranged in string configuration.
